(19) United States
(12) Patent Application Publication
(10) Pub. No.: US 2026/0083443 A1
(43) Pub. Date: Mar. 26, 2026

(54) LIMITING AND SETTING THE MAXIMUM SPEED OF BATTERY-POWERED SURGICAL INSTRUMENTS

(71) Applicant: ConMed Corporation, Largo, FL (US)

(72) Inventor: Capri Gordon, New Port Richey, FL (US)

(73) Assignee: ConMed Corporation, Largo, FL (US)

(21) Appl. No.: 19/329,119

(22) Filed: Sep. 15, 2025

Related U.S. Application Data
(60) Provisional application No. 63/694,497, filed on Sep. 13, 2024.

(51) Int. Cl. A61B 17/00; G16H 40/63

(52) U.S. Cl. CPC A61B 17/00; G16H 40/63; A61B 2017/00199; A61B 2017/00398; A61B 2017/00734

(57) ABSTRACT

A cordless surgical instrument that is configured to let users set and limit a maximum speed for safer and more precise operation. The tool features a handpiece, a battery, and a control mechanism, along with a special mode that allows the user to program the maximum speed. The instrument includes a processor and a non-transitory memory storing program instructions executable to record a maximum speed set by the user and to limit the speed of the surgical accessory to the recorded maximum during subsequent operation, regardless of the extent of actuation of the control mechanism. Once set, the tool will not exceed the maximum speed. The system uses built-in electronics to record and enforce the maximum speed maximum.

### CROSS-REFERENCE TO RELATED APPLICATIONS

The present application claims priority to U.S. Provisional Application No. 63/694,497, filed on Sep. 13, 2024, hereby incorporated by reference in its entirety.

### BACKGROUND OF THE INVENTION

### 1. Field of the Invention

The present disclosure relates to battery-powered surgical instruments and, more particularly, to battery-powered surgical instruments, the maximum speed of each is programmed, structured and/or configured to be limited and set.

### 2. Description of Related Art

Conventional motorized/powered surgical instruments including corded and battery-operated surgical instruments are well known in the art. Such surgical instruments are used during orthopedic surgical procedures (including, but not limited to, tissue resection procedures) and can include drills, saws, shavers and burs. The speed at which the drills, saws, shavers and burs operate can be varied.

Limiting and setting the maximum speed on battery-powered surgical instruments can be a very desirable feature since different speeds can be more optimal for certain surgical applications. Furthermore, certain battery-powered surgical instruments having, e.g., drill bits, shaver blades, burs, and saw blades, can have better performances at different speeds. Many powered surgical instruments thus have variable speed activation controls (triggers, sliders, buttons, levers or foot pedals), i.e., the speed of the instrument varies as the control mechanism is depressed. However, controlling or maintaining a certain speed with a variable control can be difficult, and surgeons prefer the ability to fully depress the control mechanism and know that it is at a desired and/or optimal speed for the surgical application and/or the particular instrument in use.

Most powered surgical instruments are directly connected to a drive console (i.e. corded) provide the ability for the user to set the maximum speed as well as display the speed on the console. Thus, when the surgeon depresses the control mechanism on the surgical handpiece to its fullest extent, the maximum desired speed is obtained. However, this ability is currently not available for battery-powered (cordless) surgical instruments. Accordingly, there is a need in the art for an approach that can program, structure, and/or the maximum speed for battery-powered surgical instruments Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

It is therefore a principal object and advantage of embodiments of the present disclosure to provide a battery-powered surgical instrument that eliminates one or more of the problems/issues/deficiencies associated with conventional battery-powered surgical instruments. In particular, the present disclosure is directed to inventive battery-powered surgical instrument embodiments programmed, structured and/or configured to limit and set a maximum speed of the instrument. The exemplary embodiments of the battery-powered surgical instrument are provided with the ability to set a new speed limit on the instrument such that the instrument will not operate faster than the speed set by the user/surgeon even if the surgeon fully actuates the control mechanism, such as a trigger, slider, lever, footswitch pedal, etc.

In an embodiment, the surgical instrument includes a handpiece, a distal mount for releasably attaching a surgical accessory, a battery, a control mechanism for actuating the accessory, a mode switch with multiple user-selectable positions, including a programming mode, and a processor that, when the mode switch is in programming mode, records the maximum speed of the surgical accessory as set by the user via the control mechanism and subsequently limits the accessory's speed to that recorded maximum during any later operation. For example, in various implementations, programming mode may be entered via a distinct switch position separate from forward, reverse, or oscillating operation. The maximum speed may be recorded when the control mechanism returns to a home position, when the accessory speed is maintained for a predetermined duration, or when the mode switch is changed out of programming mode. A user interface element, such as a switch, slider, knob, or potentiometer, may be provided to set or adjust the maximum speed and the processor may allow resetting the maximum speed by re-entering programming mode.

In another embodiment, a method involves limiting the maximum speed of a surgical instrument. The method includes actuating a mode switch to enter programming mode, operating a control mechanism to accelerate a surgical accessory to a desired speed, recording the peak speed reached, setting that peak speed as a maximum speed limit, and restricting the speed to the set limit during subsequent operations. In additional implementations, the method may involve displaying the set maximum speed to the user, resetting the limit when programming mode is entered again, recording the limit upon the return of the control mechanism to its home position, recording upon maintaining the speed for a predetermined time, recording upon switching from programming mode to another operational mode, and providing a user interface element, such as a switch, slider, knob, or potentiometer, for user-driven speed setting.

These and other aspects of the disclosure will be apparent from and elucidated with reference to the embodiment(s) described hereinafter.

### BRIEF DESCRIPTION OF THE SEVERAL VIEWS OF THE DRAWING(S)

The present disclosure will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a side perspective schematic representation of a battery-powered surgical instrument, according to an embodiment;

FIG. 2 is a side perspective schematic representation of a battery-powered surgical instrument, according to another embodiment;

FIG. 3 is a schematic of a circuit board for a battery-powered surgical instrument, according to an embodiment

FIG. 3 is a flowchart illustrating a method of using battery-powered surgical instrument, according to an embodiment;

FIG. 4 is a flowchart illustrating a method of using battery-powered surgical instrument, according to another embodiment;

FIG. 5 is a flowchart illustrating a method of using battery-powered surgical instrument, according to another embodiment;

FIG. 6 is a flowchart illustrating a method of using battery-powered surgical instrument, according to another embodiment;

FIG. 7 is a flowchart of a method for limiting the maximum speed of a battery-powered surgical instrument, according to another embodiment; and

FIG. 8 is a flowchart of a method for limiting the maximum speed of a battery-powered surgical instrument.

### DETAILED DESCRIPTION OF THE INVENTION

Aspects of the present disclosure and certain features, advantages, and details thereof, are explained more fully below with reference to the non-limiting examples illustrated in the accompanying drawings. Descriptions of well-known structures are omitted so as not to unnecessarily obscure the disclosure in detail. It should be understood, however, that the detailed description and the specific non-limiting examples, while indicating aspects of the disclosure, are given by way of illustration only, and are not by way of limitation. Various substitutions, modifications, additions, and/or arrangements, within the spirit and/or scope of the underlying inventive concepts will be apparent to those skilled in the art from this disclosure.

While embodiments of the present disclosure have been particularly shown and described with reference to certain exemplary embodiments, it will be understood by one skilled in the art that various changes in detail may be affected therein without departing from the spirit and scope of the disclosure as defined by claims that can be supported by the written description and drawings. Further, where exemplary embodiments are described with reference to a certain number of elements or number/order of steps it will be understood that the exemplary embodiments can be practiced utilizing either less than or more than the certain number of elements or number/order of steps. If elements are shown in a particular Figure discussed below are not specifically identified with respect to that Figure, the elements should be sufficiently identified with respect to at least one other Figure.

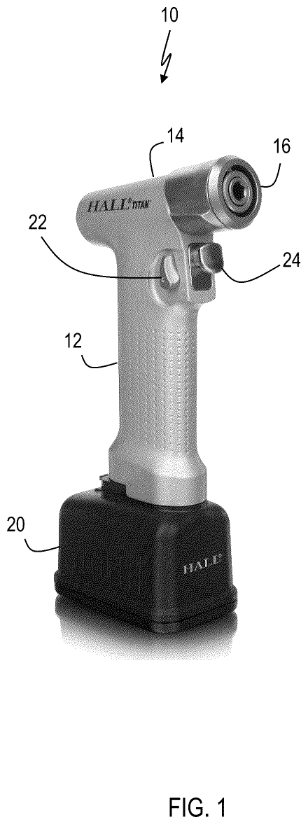

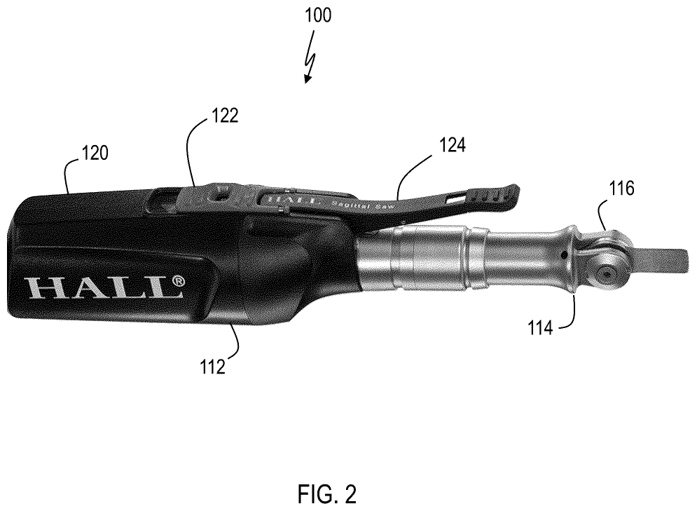

Referring now to the figures, wherein like reference numerals refer to like parts throughout, FIGS. 1 and 2 show side perspective view schematic representations of a first exemplary battery-powered (cordless) surgical instrument 10 and a second exemplary battery-powered (cordless) surgical instrument 100, respectively.

Referring to FIG. 1, battery-powered surgical instrument 10 includes a handpiece 12 having a distal end 14 having a mount 16 to which various drill bits, shaver blades, burs, and saw blades (not shown) can be releasably attached as desired. Battery-powered surgical instrument 10 includes a battery 20, a mode switch 22 having several user-selectable positions, and a control mechanism 24 for actuating a motor within battery-powered surgical instrument 10 to drive shaver blade or any other surgical implement attached thereto.

Referring to FIG. 2, battery-powered surgical instrument 100 has a pencil-grip handpiece 12 having a distal end 114 with a mount 118 shown with a shaver blade attached as an example. As with battery-powered surgical instrument 10, various drill bits, burs, and saw blades may be attached as desired. Battery-powered surgical instrument 100 includes a battery 120, a mode switch 122 having several user-selectable positions, and a control mechanism 124 for actuating a motor within battery-powered surgical instrument 100 to drive shaver blade or any other surgical implement attached thereto.

Mode switch 22, 122 allows battery-powered surgical instrument 10 and battery-powered surgical instrument 100 to switch to and operate in different modes, such as safe, forward, reverse, oscillate. In certain cases, mode switch 22, 122 can provide two different speeds of operation, such as a “standard” speed that is slower and a “fast” speed that is faster than the standard speed.

In accordance with an embodiment of the present disclosure, mode switch 22, 122 (or a separate switch, button or other actuator) can be actuated to a different or distinct position to enable a programming mode that is configured to allow a user to set a maximum speed of the instrument. In the programming mode, a user can set a maximum speed limit, and then each time control mechanism 24, 124, e.g., a trigger, lever, slider, or foot pedal, etc., is fully depressed, the instrument is prohibited from operating any faster than the maximum speed limit set in programming mode.

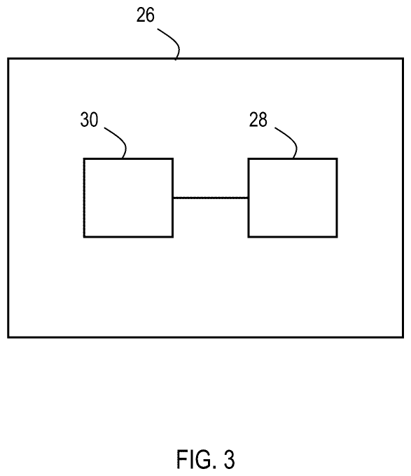

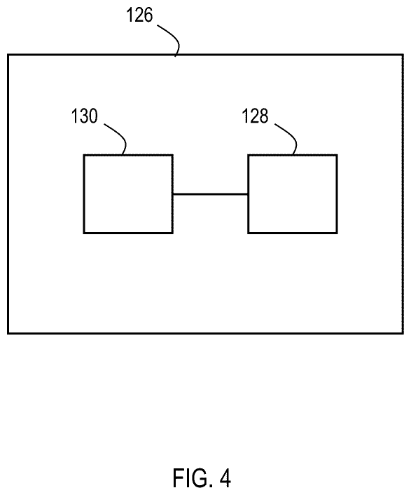

This ability to set a maximum speed limit is programming mode may be implemented via software or firmware and saved in a memory. As seen in FIG. 3, battery-powered (cordless) surgical instrument 10 can include a circuit board 26 having a non-transitory storage medium (i.e., memory) 28 configured to store a plurality of program instructions, and a processor 30 configured to execute the program instructions stored in the memory. Further, printed circuit board 26 may be configured to control battery management functions and motor control (of an attached or otherwise included motor), as well as other operations and features. As seen in FIG. 4, battery-powered (cordless) surgical instrument 100 can each respectively include circuit board 126 having a non-transitory storage medium (i.e., memory) 128 configured to store a plurality of program instructions, and a processor 130 configured to execute the program instructions stored in the memory. Further, printed circuit board 126 may be configured to control battery management functions and motor control (of an attached or otherwise included motor), as well as other operations and features.

Limiting the maximum speed of battery-powered (cordless) surgical instrument 10 and battery-powered (cordless) surgical instrument 100 using mode switch 22, 122 (or a separate mode switch, button or other actuator) can be implemented via many different methods, as should be appreciated by those of ordinary skill in the art in conjunction with a review of this disclosure, examples of which are described herein below. The steps of the methods can be accomplished by mode switch 22, 122 and one or more processors executing program code stored in one or more non-transitory storage media such as those associated with circuit board 26, 126.

Advantages of the disclosure are illustrated by the following Examples. However, the conditions and details of the steps are to be interpreted to apply broadly in the art and should not be construed to unduly restrict or limit the embodiments of this disclosure in any way.

### Example 1

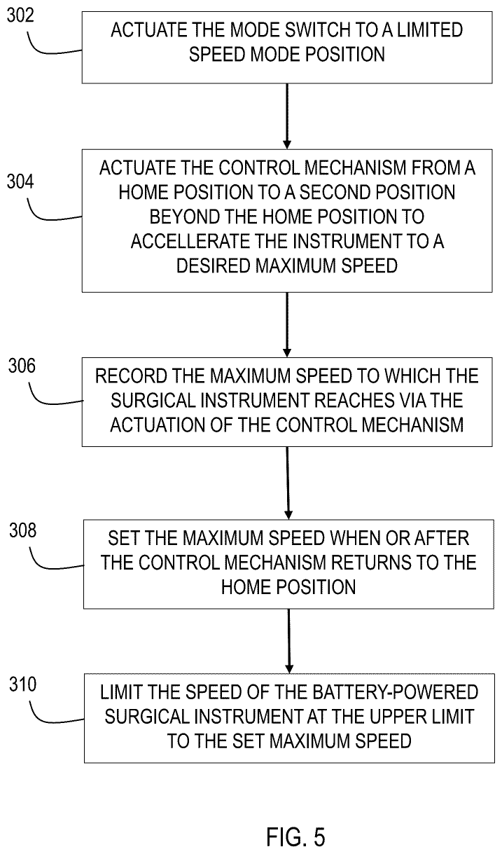

Referring to FIG. 5, there is shown a flowchart of a method 300 for limiting the maximum speed of a battery-powered surgical instrument. At step 302, a user actuates the mode switch to a new mode position (i.e., limited speed mode). At step 304, the user actuates the control mechanism from a rest/home position to a second position beyond the home position to accelerate the battery-powered surgical instrument up to a desired maximum speed. At step 306, the software/firmware is programmed to record and records the highest/maximum speed to which the surgical instrument reaches via the actuation of the control mechanism. At step 308, the maximum speed is set when or after the control mechanism returns to the home position. At step 310, when the user actuates the control mechanism again, the speed of the battery-powered surgical instrument is limited at the upper limit to the set maximum speed.

### Example 2

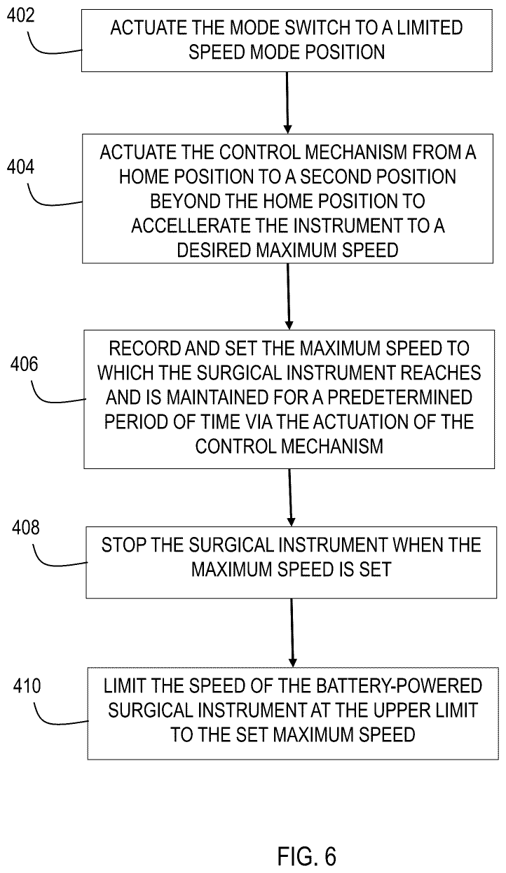

Referring to FIG. 6, there is shown a flowchart of a method 400 for limiting the maximum speed of a battery-powered surgical instrument. At step 402, a user actuates the mode switch to a new mode position (i.e., limited speed mode). At step 404, the user actuates the control mechanism from a rest/home position to a second position beyond the home position to accelerate the battery-powered surgical instrument up to a desired maximum speed. At step 406, the software/firmware is programmed to record/set and records/sets a maximum speed limit as the speed to which the surgical instrument reaches and is maintained for a predetermined period of time via the actuation of the control mechanism. At step 408, the surgical instrument stops running when the maximum speed is set. At step 410, when the user actuates the control mechanism again, the speed of the battery-powered surgical instrument is limited at the upper limit to the set maximum speed.

### Example 3

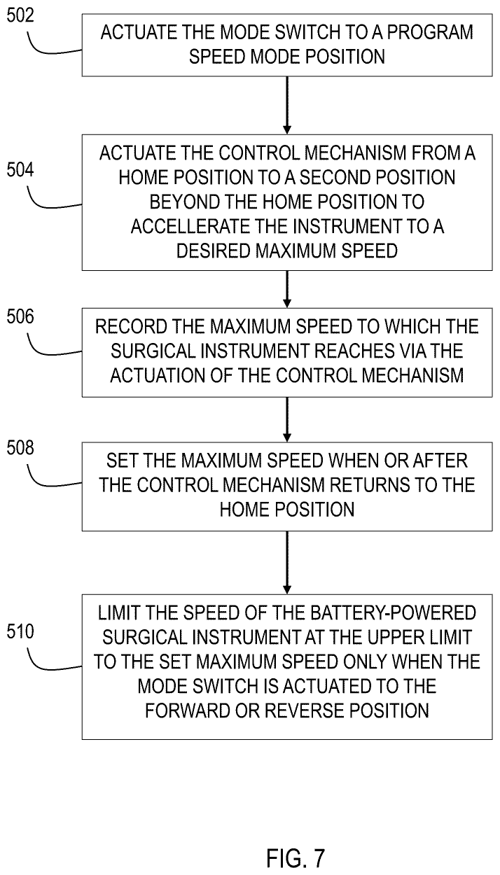

Referring to FIG. 7, there is shown a flowchart of a method 500 for limiting the maximum speed of a battery-powered surgical instrument. At step 502, a user actuates the mode switch to a new mode position (i.e., program speed mode). At step 504, the user actuates the control mechanism from a rest/home position to a second position beyond the home position to accelerate the battery-powered surgical instrument up to a desired maximum speed. At step 506, the software/firmware is programmed to record and records the highest/maximum speed to which the surgical instrument reaches via the actuation of the control mechanism. At step 508, the maximum speed is set when or after the control mechanism returns to the home position. At step 510, when the user actuates the control mechanism again, the speed of the battery-powered surgical instrument is limited at the upper limit to the set maximum speed only when the mode switch is actuated to the forward or the reverse position. The maximum speed can be reset by actuating the mode switch to the new mode position and performing the steps in FIG. 5 over again.

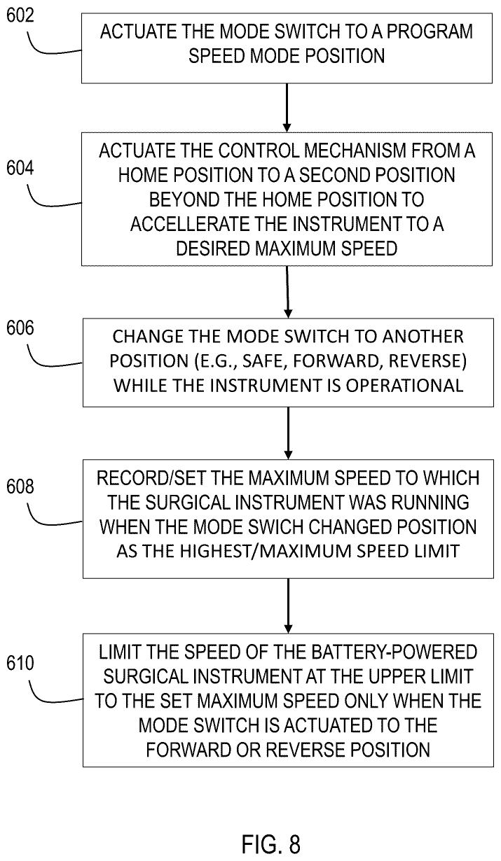

Referring to FIG. 8, there is shown a flowchart of a method 600 for limiting the maximum speed of a battery-powered surgical instrument. At step 602, a user actuates the mode switch to a new mode position (i.e., program speed mode). At step 604, the user actuates the control mechanism from a rest/home position to a second position beyond the home position to accelerate the battery-powered surgical instrument up to a desired maximum speed (which can be passed). At step 606, the user changes the mode switch to another position (e.g., safe, forward, reverse) while the instrument is operational (or actuates another switch, lever, button). At step 608, the software/firmware is programmed to record/set and records/sets the speed that the instrument was running when the mode switch changed position as the highest/maximum speed limit. At step 610, the user switches to another mode position such as “forward” or “reverse,” and the speed of the battery-powered surgical instrument is limited at the upper limit to the set maximum speed in forward or reverse respectively.

In accordance with an alternative embodiment, a display/screen or another switch/slider/knob (similar to a potentiometer) can be added to a battery-powered surgical instrument that can allow control of (or otherwise limit) the speed of the instrument.

While embodiments of the present disclosure have been particularly shown and described with reference to certain exemplary embodiments, it will be understood by one skilled in the art that various changes in detail may be affected therein without departing from the spirit and scope of the disclosure as defined by claims that can be supported by the written description and drawings. Further, where exemplary embodiments are described with reference to a certain number of elements it will be understood that the exemplary embodiments can be practiced utilizing either less than or more than the certain number of elements.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts and equivalents of all means or step plus function elements in the claims below, if any, are intended to include any structure, material or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of one or more aspects of the disclosure and the practical application, and to enable others of ordinary skill in the art to understand one or more aspects of the present disclosure for various embodiments with various modifications as are suited to the particular use contemplated.

What is claimed is:

1. A surgical instrument comprising:
a handpiece;
a mount positioned at a distal end of the handpiece and configured to releasably attach a surgical accessory;
a battery operably connected to the handpiece;
a control mechanism configured to actuate the surgical accessory;
a mode switch having a plurality of user-selectable positions, wherein at least one position is a programming mode; and
a processor programmed to record a maximum speed of the surgical accessory as set by a user via the control mechanism when the mode switch in the position of the programming mode and to limit a speed of the surgical accessory to the maximum speed set by the using during any subsequent operation of the control mechanism.

2. The surgical instrument of claim 1, wherein the programming mode is enabled by actuating the mode switch to a distinct position that is separate from another position that will allow operation of the surgical instrument in one or more of a forward direction, a reverse direction, and an oscillating direction.

3. The surgical instrument of claim 1, wherein the maximum speed is set by the processor when the control mechanism is returned to a home position after being actuated to accelerate the surgical accessory.

4. The surgical instrument of claim 1, wherein the maximum speed is set by the processor when the speed of the surgical accessory is maintained for a predetermined period of time.

5. The surgical instrument of claim 1, wherein the maximum speed is set by the processor when the mode switch is changed from the programming mode to another operational mode while the surgical accessory is being operated.

6. The surgical instrument of claim 1, further comprising a user interface element selected from the group consisting of a switch, slider, knob, and a potentiometer that is configured to allow the user to set the maximum speed.

7. The surgical instrument of claim 1, wherein the processor is further configured to allow resetting of the maximum speed by re-entering the programming mode via the mode switch.

8. The surgical instrument of claim 1, wherein the surgical accessory is selected from the group consisting of a drill bit, a shaver blade, a bur, and a saw blade.

9. The surgical instrument of claim 1, wherein processor is associated with a non-transitory memory containing program instructions.

10. The surgical instrument of claim 9, wherein the processor is programmed to store the maximum speed in the non-transitory memory.

11. A method of limiting a maximum speed of a surgical instrument, comprising:
actuating a mode switch to a programming mode;
actuating a control mechanism to accelerate a surgical accessory that is powered by a batter to a desired speed;
recording the desired speed reached by the surgical accessory as a maximum speed;
setting the maximum speed as a maximum speed limit; and
limiting the speed of the surgical accessory to the maximum speed limit during any subsequent operation.

12. The method of claim 11, further comprising displaying the set maximum speed to a user.

13. The method of claim 11, further comprising resetting the maximum speed when the mode switch is actuated to the programmed mode a second time.

14. The method of claim 11, wherein the maximum speed is set when the control mechanism is returned to a home position after actuation by a user.

15. The method of claim 11, wherein the maximum speed is set when the speed of the surgical accessory is maintained for a predetermined period of time.

16. The method of claim 11, wherein the maximum speed is set when the mode switch is changed from the programming mode to another operational mode while the surgical accessory is running.

17. The method of claim 11, further comprising providing a user interface element selected from the group consisting of a switch, slider, knob, and a potentiometer, to allow a user to set the maximum speed.

18. The method of claim 11, wherein the step of recording the maximum speed reached by the surgical accessory comprises a processor of the surgical instrument storing the maximum speed in a non-transitory memory associated with the processor.
